(19) United States
(12) Patent Application Publication
(10) Pub. No.: US 2016/0256103 A1
(43) Pub. Date: Sep. 8, 2016

(54) WEARABLE MEDICAL EXAMINATION AND TREATMENT DEVICE AND WEARABLE MEDICAL EXAMINATION AND TREATMENT SYSTEM COMPRISING THE SAME

(71) Applicant: AETO TECHNOLOGY CORP, TAIPEI CITY (TW)

(72) Inventors: SHU-WEI CHANG, TAIPEI CITY (TW); HSIN-CHENG LAI, NEW TAIPEI CITY (TW); HO-TENG CHANG, NEW TAIPEI CITY (TW)

(21) Appl. No.: 14/700,225

(22) Filed: Apr. 30, 2015

(30) Foreign Application Priority Data
Mar. 3, 2015 (TW) 104106591

(51) Int. Cl. A61B 5/00; H04W 4/00; A61N 1/36; A61H 39/00; A61N 1/04; H04B 1/3827; A61B 5/053

(52) U.S. Cl. CPC A61B 5/4836; H04B 1/385; H04W 4/005; A61B 5/6806; A61B 5/053; A61B 5/0004; A61B 5/742; A61B 5/6807; A61B 5/7203; A61H 39/002; A61N 1/0484; A61N 1/36014; A61B 2562/0247; A61H 2201/1635; A61H 2201/164

(57) ABSTRACT

A wearable medical examination and treatment device and system are introduced. The wearable medical examination and treatment device includes an enclosing unit, test electrode unit, ground electrode, processing module and transmission module which are disposed at the enclosing unit. The enclosing unit encloses a human body part of a subject and is fixed thereto by positioning holes disposed at the enclosing unit. The test electrode unit and ground electrode are in contact with acupoint testing points of the human body part. The processing module is electrically connected to the test electrode unit and ground electrode and sends testing and treating electrical signals according to testing and treating instructions. The transmission module is electrically connected to the processing module to send impedance signals to an external electronic device so as for the subject's physiological information to be displayed on the electronic device according to the impedance signals.

### CROSS-REFERENCE TO RELATED APPLICATION

This non-provisional application claims priority under 35 U.S.C. §119(a) on Patent Application No(s).104106591 filed in Taiwan, R.O.C. on Mar. 3, 2015, the entire contents of which are hereby incorporated by reference.

### FIELD OF TECHNOLOGY

The present invention relates to medical examination and treatment devices and wearable medical examination and treatment systems comprising the devices, and more particularly, to a wearable medical examination and treatment device and a medical examination and treatment system comprising the device, for use in assessing meridians to thereby gather physiological information.

### BACKGROUND

Due to medical advancement, human physiological information can be gathered with a non-invasive meridian instrument. The meridian instrument comprises test electrodes adapted to be in contact with a human body, send electrical signals, such as current signals or voltage signals, to the human body, so as to assess the impedance and electric potential difference at acupoint testing points on the human body. By assessing the impedance and electric potential difference at the acupoint testing points on the human body, it is feasible to evaluate the physiological state of the human body.

However, a typical conventional meridian instrument is equipped with just one single test electrode, and in consequence the conventional meridian instrument examines the acupoint testing points of a subject's body one by one in a time-consuming manner. Furthermore, the conventional meridian instrument is effective in evaluating the physiological state of the human body but is not designed to give medical treatment to the subject diagnosed with any abnormal physiological state. Hence, there is still room for improvement in the conventional meridian instrument.

### SUMMARY

In view of the aforesaid drawbacks of the prior art, it is an objective of the present invention to provide a meridian instrument capable of examining multiple acupoint testing points.

Another objective of the present invention is to provide a meridian instrument which serves both a diagnostic purpose and a therapeutic purpose.

In order to achieve the above and other objectives, the present invention provides a wearable medical examination and treatment device and a wearable medical examination and treatment system which comprises the device.

The wearable medical examination and treatment device comprises an enclosing unit, a test electrode unit, a ground electrode, a processing module, and a transmission module. The enclosing unit has positioning holes which penetrate the enclosing unit. The enclosing unit encloses a human body part of a subject and is fixed to the human body part by the positioning holes. The test electrode unit has a plurality of test electrodes. The test electrodes are disposed at predetermined acupoint testing positions in the enclosing unit, respectively, and thus are in contact with the acupoint testing points of the human body part, respectively. The predetermined acupoint testing positions are aligned with the acupoint testing points of the human body part. The ground electrode is disposed in the enclosing unit and is in contact with the human body part. The processing module is disposed at the enclosing unit, electrically connected to the test electrode unit and the ground electrode, and adapted to generate and send a testing electrical signal to the test electrode unit in accordance with a testing instruction, to allow the test electrode unit, the human body part and the ground electrode to thereby form a circuit loop and thus measure the impedance of the acupoint testing points, or is adapted to generate and send a treating electrical signal to the test electrode unit in accordance with a treating instruction to thereby stimulate the acupoint testing points. The transmission module is disposed at the enclosing unit, electrically connected to the processing module, and adapted to send the impedance-related data to an external electronic device such that the electronic device displays a physiological information of the subject in accordance with the impedance-related data.

In an embodiment of the present invention, the enclosing unit is glove-shaped and corresponds in shape to the human body part which is a hand. The glove-shaped enclosing unit encloses the hand but allows the fingers of the hand to penetrate the positioning holes of the glove-shaped enclosing unit, respectively, and thus be exposed from the glove-shaped enclosing unit.

In an embodiment of the present invention, the enclosing unit is sock-shaped and corresponds in shape to the human body part which is a foot. The sock-shaped enclosing unit encloses the foot but allow portions of the foot to penetrate the positioning holes of the sock-shaped enclosing unit, respectively, and thus be exposed from the sock-shaped enclosing unit.

In an embodiment of the present invention, the test electrode unit of the wearable medical examination and treatment device further comprises a plurality of pressure sensing modules connected to the test electrodes, respectively, and adapted to sense a downward pressure when in contact with the acupoint testing points and send a pressure correction signal corresponding to the downward pressure to the processing module so as to correct the impedance-related data.

In an embodiment of the present invention, the test electrodes are replaceable button electrodes.

In an embodiment of the present invention, the transmission module is a wireless transmitter.

In an embodiment of the present invention, the acupoint testing points correspond in position to the Twelve Source Points described in Yellow Emperor's Inner Canon, respectively.

The wearable medical examination and treatment system is in wireless communication with an electronic device disposed outside and comprises the wearable medical examination and treatment device. The wearable medical examination and treatment system comprises the wearable medical examination and treatment device, generates impedance-related data pertaining to a subject's physiological state in accordance with a testing instruction, and sends the impedance-related data pertaining to the subject's physiological state to the electronic device. Alternatively, the wearable medical examination and treatment system sends a treating electrical signal in accordance with a treating instruction so as to stimulate the subject's acupoint testing point. The wearable medical examination and treatment system further comprises a cloud server for receiving the impedance signals through the electronic device, converting the impedance signals into a physiological information, and sending the physiological information to the electronic device so as for the physiological information to be displayed on a display module of the electronic device.

Accordingly, the present invention provides a wearable medical examination and treatment device and a wearable medical examination and treatment system which allow a subject's multiple acupoint testing points to be examined simultaneously and are capable of performing treatment on the subject with an enclosing unit, a test electrode unit, a ground electrode, a processing module and a transmission module.

### BRIEF DESCRIPTION

Objectives, features, and advantages of the present invention are hereunder illustrated with specific embodiments in conjunction with the accompanying drawings, in which:

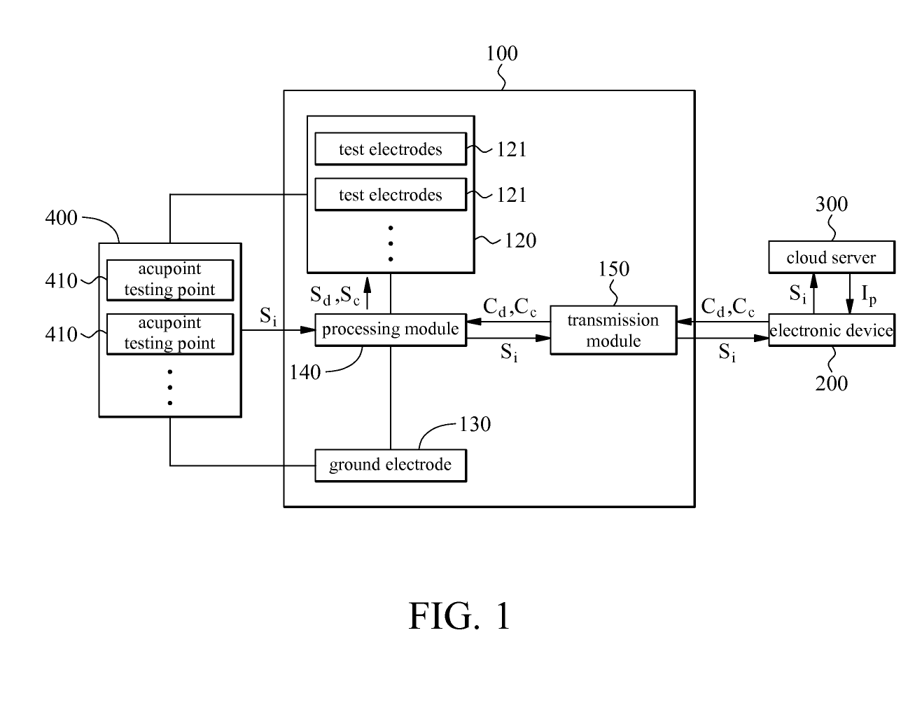

FIG. 1 is a function block diagram of a wearable medical examination and treatment device, an electronic device, and a cloud server according to the first aspect and the second aspect of an embodiment of the present invention;

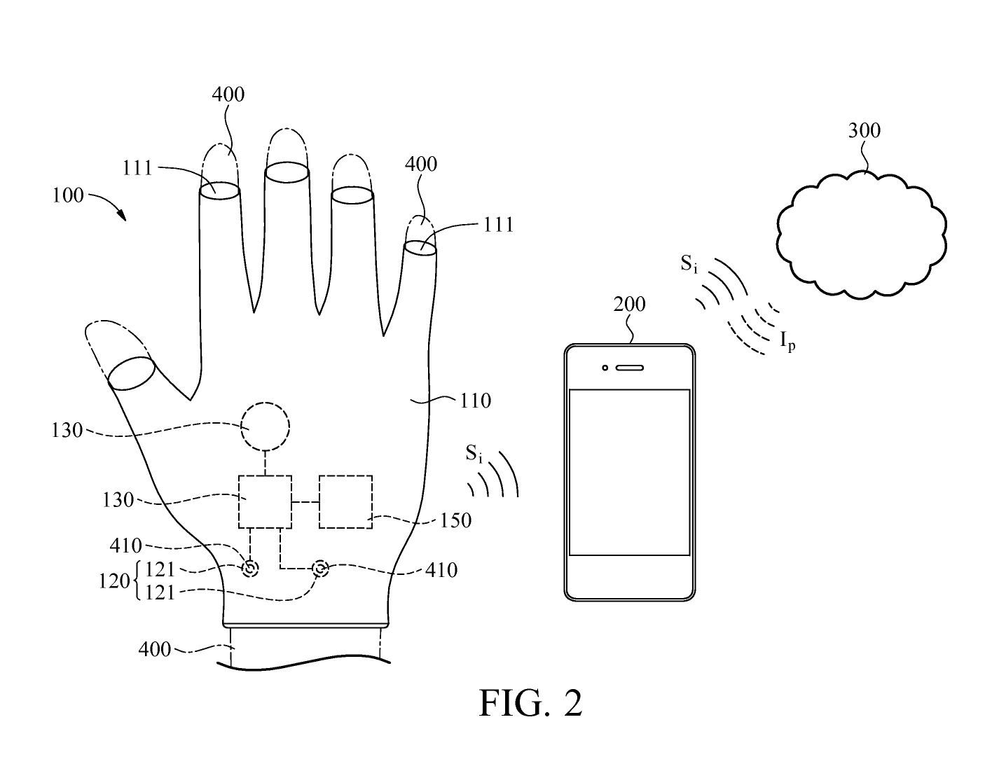

FIG. 2 is a perspective view of the wearable medical examination and treatment device, the electronic device and the cloud server according to the first aspect of an embodiment of the present invention;

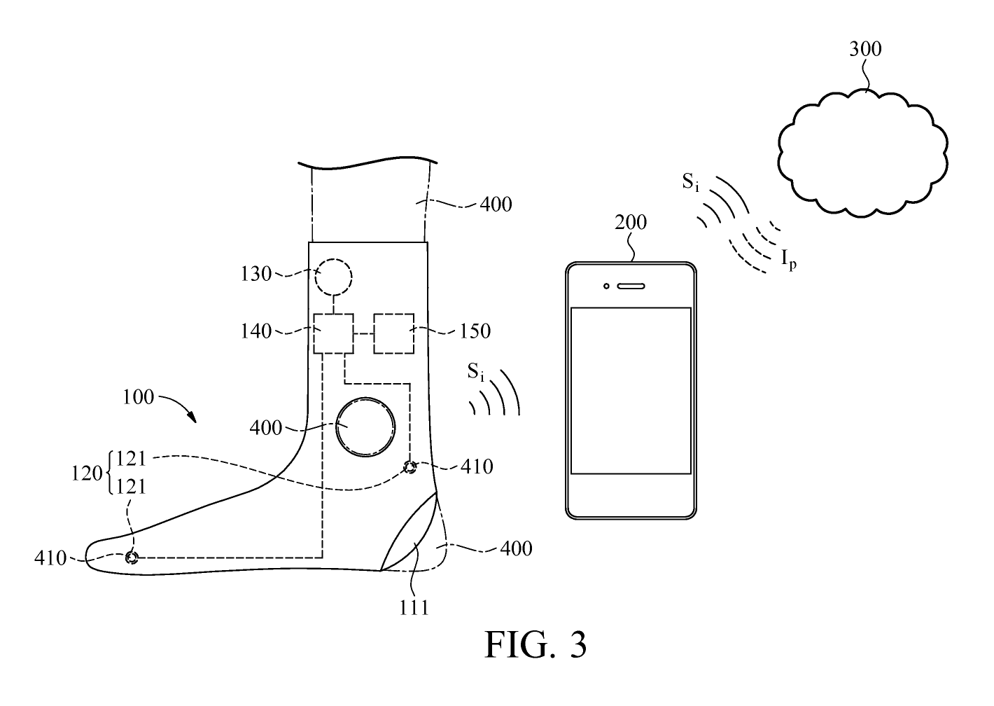

FIG. 3 is a perspective view of the wearable medical examination and treatment device, the electronic device and the cloud server according to the second aspect of an embodiment of the present invention;

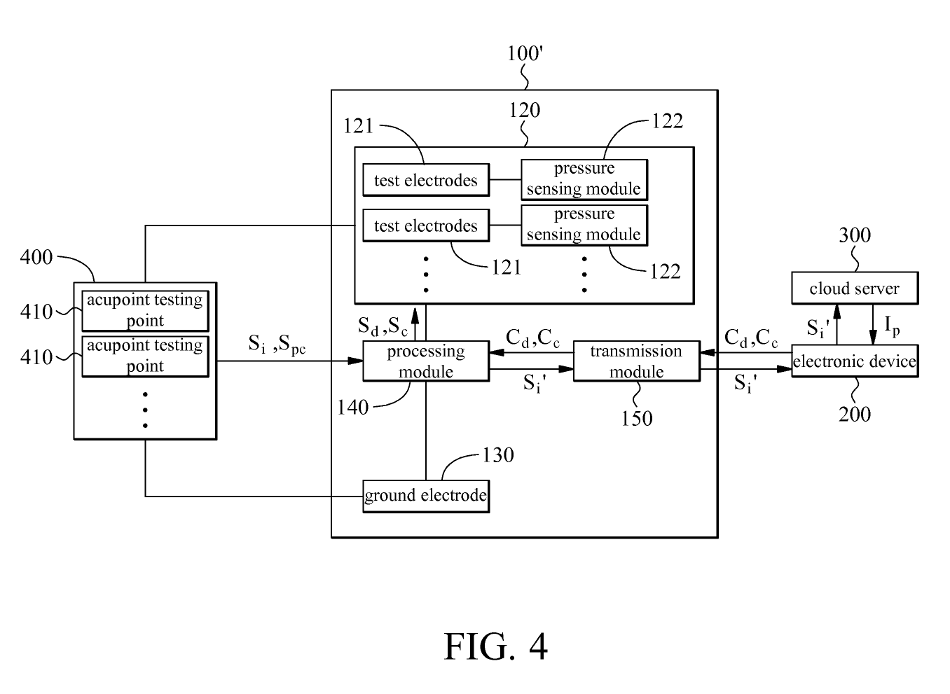

FIG. 4 is a function block diagram of a wearable medical examination and treatment device, the electronic device, and the cloud server according to another embodiment of the present invention;

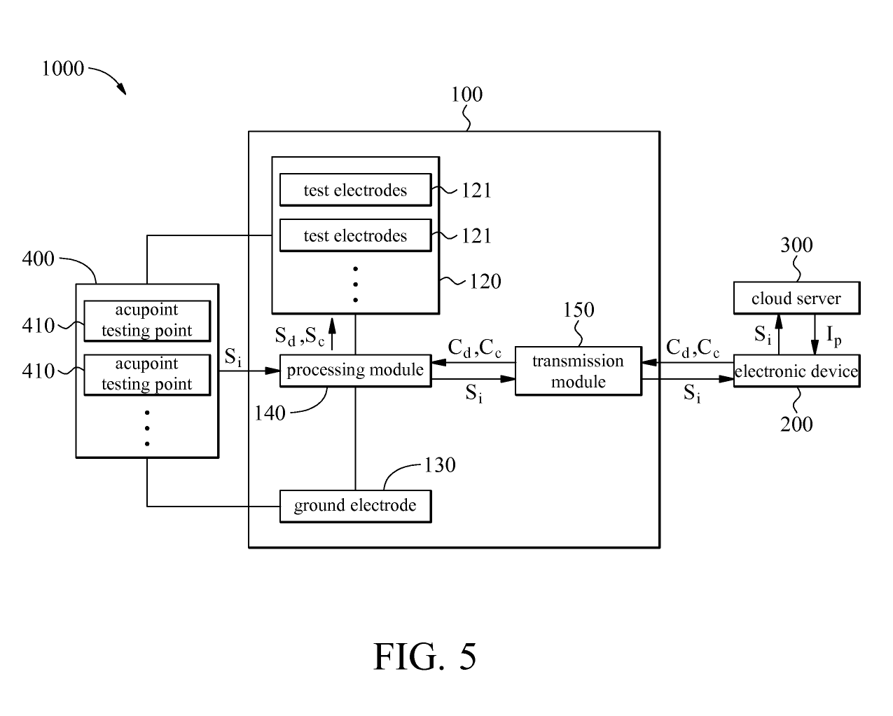

FIG. 5 is a function block diagram of the wearable medical examination and treatment system and the electronic device according to the first aspect of an embodiment of the present invention; and

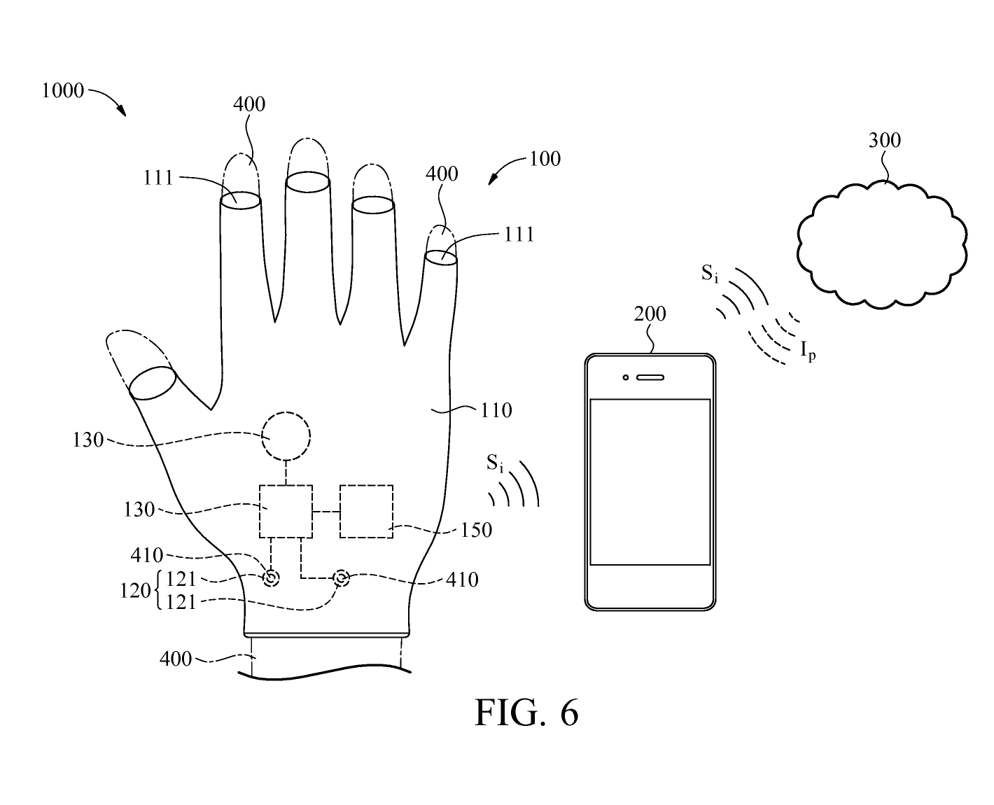

FIG. 6 is a perspective view of the wearable medical examination and treatment system and the electronic device according to the first aspect of an embodiment of the present invention.

### DETAILED DESCRIPTION

FIG. 1 is a function block diagram of a wearable medical examination and treatment device 100, an electronic device 200 and a cloud server 300 according to the first aspect and the second aspect of an embodiment of the present invention. FIG. 2 is a perspective view of the wearable medical examination and treatment device 100, the electronic device 200 and the cloud server 300 according to the first aspect of an embodiment of the present invention. FIG. 3 is a perspective view of the wearable medical examination and treatment device 100, the electronic device 200 and the cloud server 300 according to the second aspect of an embodiment of the present invention.

The wearable medical examination and treatment device 100 comprises an enclosing unit 110, a test electrode unit 120, a ground electrode 130, a processing module 140, and a transmission module 150.

The enclosing unit 110 has a plurality of positioning holes 111 for penetrating the enclosing unit 110. The enclosing unit 110 is for use in enclosing a human body part 400 of a subject and fixed to the human body part 400 by the positioning holes 111. In this embodiment, the enclosing unit 110 is provided in two aspects shown in FIG. 2 and FIG. 3, respectively.

Referring to FIG. 2, the enclosing unit 110 is glove-shaped and corresponds in shape to the human body part 400. In the first aspect of this embodiment of the present invention, the human body part 400 is a hand exemplified by the right hand. The enclosing unit 110 encloses the hand but allows the five fingers of the hand to penetrate the positioning holes 111, respectively, and be exposed from the enclosing unit 110 such that the enclosing unit 110 can be fixed to the hand.

Referring to FIG. 3, the enclosing unit 110 is sock-shaped and corresponds in shape to the human body part 400. In the second aspect of this embodiment of the present invention, the human body part 400 is a foot exemplified by the right foot. The enclosing unit 110 encloses the foot but allows portions of the foot to penetrate the positioning holes 111, respectively, and be exposed from the enclosing unit 110 such that the enclosing unit 110 can be fixed to the foot.

Although the glove-shaped enclosing unit 110 is shown in FIG. 2 and the sock-shaped enclosing unit 110 is shown in FIG. 3, such technical features are illustrative rather than restrictive of the present invention. For example, if the human body part 400 is a waist, the enclosing unit 110 will be waist-shaped.

The test electrode unit 120 has a plurality of test electrodes 121. The test electrodes 121 are located at predetermined acupoint testing positions within the enclosing unit 110, respectively, and thus in contact with acupoint testing points 410 of the human body part 400. To this end, the predetermined acupoint testing positions are aligned with the acupoint testing points 410 of the human body part 400. The acupoint testing points 410 correspond in quantity and position to Twelve Source Points described in the Yellow Emperor's Inner Canon, respectively. The Twelve Source Points feature a lower impedance than acupoints in the vicinity of the Twelve Source Points and thus the Twelve Source Points manifest relatively high conductivity. Hence, the Twelve Source Points are preferably the acupoint testing points 410 according to the present invention. Once the test electrode unit 120 comes into contact with the acupoint testing points 410 and applies the electrical signals thereto, the test electrode unit 120 can measure an appropriate impedance level conductive to evaluating the physiological state of the subject. In this regard, the impedance level will be appropriate if it is relatively low, because it is difficult to evaluate the physiological state of the subject if the impedance level is high. The acupoint testing points 410 are illustrative rather than restrictive of the present invention. In a preferred embodiment, the test electrodes 121 are replaceable button electrodes; hence, the test electrodes 121 is changeable as needed. For instance, if any of the test electrodes 121 of the test electrode unit 120 is out of order, only the malfunctioning test electrode 121 will be changed.

The ground electrode 130 is disposed at the enclosing unit 110 and in contact with a human skin of the human body part 400 such that the human skin of the human body part 400 functions as a ground end of the circuit loop.

The processing module 140 is disposed either inside or outside the enclosing unit 110 and electrically connected to the test electrode unit 120 and the ground electrode 130. The processing module 140 generates a testing electrical signal Sd, such as a current signal, in accordance with a testing instruction Cd and sends the testing electrical signal Sd to the test electrode unit 120, so as for the test electrode unit 120, the human body part 400 and the ground electrode 130 to form the circuit loop for use in measuring the impedance at the acupoint testing points 410. In practice, the impedance thus measured at the acupoint testing points 410 is expressed in the form of a signal known as impedance signal Si. The testing instruction Cd is generated by the subject or a check-up technician with the processing module 140, the electronic device 200, and the cloud server 300. The processing module 140 further generates a treating electrical signal Sc, such as a current signal, in accordance with a treating instruction Cc and sends the treating electrical signal Sc to the test electrode unit 120 so as to stimulate the acupoint testing points 410. In this regard, the treating instruction Cc is generated by the subject or the check-up technical with the processing module 140, the electronic device 200, and the cloud server 300. Therefore, the subject or the check-up technician exercises near-end control over a control unit (not shown) in the processing module 140 and a control unit (not shown) in the electronic device 200. The subject or the check-up technician further exercises remote control over a control unit (not shown) of the cloud server 300. By exercising control over the aforesaid control units (not shown), the subject or the check-up technician sends the testing instruction Cd and the treating instruction Cc to the processing module 140 such that the processing module 140 sends the testing electrical signal Sd and the treating electrical signal Sc.

The transmission module 150 is disposed either inside or outside the enclosing unit 110 and electrically connected to the processing module 140. The transmission module 150 sends the impedance signals Si to the electronic device 200 which is disposed outside. A wireless communication module (not shown) of the electronic device 200 sends the impedance signals Si to the cloud server 300 which is disposed outside. A conversion module (not shown) of the cloud server 300 converts the impedance signals Si into a physiological information Ip and then sends the physiological information Ip to the electronic device 200 so as for the physiological information Ip to be displayed on a display module (not shown) of the electronic device 200, thereby informing the subject and the check-up technician of the physiological information Ip. In a preferred embodiment, the transmission module 150 is a wireless transmitter, such as a Bluetooth wireless transmitter or a wi-fi wireless transmitter.

In this embodiment, the enclosing unit 110 of the wearable medical examination and treatment device 100 is glove-shaped, and the medical examination and treatment process is described below.

First, a subject inserts his or her hand into the enclosing unit 110 of the wearable medical examination and treatment device 100 and then passes the fingers of the hand through the positioning holes 111 of the enclosing unit 110 to thereby fix the enclosing unit 110 to the hand. At this point in time, the test electrodes 121 of the test electrode unit 120 are in contact with the acupoint testing points 410, such as the Twelve Source Points, of the hand of the subject, respectively, whereas the ground electrode 130 is in contact with the hand of the subject. Afterward, the subject or a check-up technician (not shown) sends the testing instruction Cd to the processing module 140 by exercising control over the processing module 140, the electronic device 200, and the cloud server 300. Then, the processing module 140 sends the testing electrical signal Sd to the test electrode unit 120 in accordance with the testing instruction Cd such that the test electrode unit 120, the human body part 400 and the ground electrode 130 together form the circuit loop for use in detecting and measuring the impedance signal Si at the acupoint testing points 410. Afterward, the transmission module 150 wirelessly transmits the impedance signals Si to the electronic device 200, and then the wireless communication module of the electronic device 200 wirelessly transmits the impedance signals Si to the cloud server 300. A conversion module (not shown) of the cloud server 300 converts the impedance signals Si into the physiological information Ip and sends the physiological information Ip to the electronic device 200. Finally, a display module (not shown) of the electronic device 200 displays the physiological information Ip to thereby inform the subject and the check-up technician of the physiological information Ip. The subject or the check-up technician exercises control over the processing module 140, the electronic device 200, and the cloud server 300 to display all or part of the physiological information Ip on the display module of the electronic device 200 as needed.

If the physiological information Ip (corresponding to the impedance signal Si at the acupoint testing points 410) is determined to be abnormal (by a conventional means) and thus treatment has to be performed on the subject, the subject or the check-up technician can perform treatment on the subject immediately with the wearable medical examination and treatment device 100, and the treatment process is described below. The subject or the check-up technician exercises control over the processing module 140, the electronic device 200, and the cloud server 300 to thereby send the treating instruction Cc to the processing module 140 such that the processing module 140 generates and sends the treating electrical signal Sc to the test electrode unit 120 to stimulate the acupoint testing points 410 of the subject with a view to restoring the subject's health.

Referring to FIG. 4 is a function block diagram of a wearable medical examination and treatment device 100′, the electronic device 200, and the cloud server 300 according to another embodiment of the present invention.

Unlike the wearable medical examination and treatment device 100, the wearable medical examination and treatment device 100′ in another embodiment is characterized in that: the test electrode unit 120 of the wearable medical examination and treatment device 100′ further comprises a plurality of pressure sensing modules 122; and the pressure sensing modules 122 are connected to the test electrodes 121, respectively, to sense a downward pressure as soon as the pressure sensing modules 122 come into contact with the acupoint testing points 410, respectively, and send a pressure correction signal Spc corresponding to the downward pressure to the processing module 140, so as to correct and turn the impedance signals Si into a correction impedance signal Si′. The pressure sensing modules 122 each have an elastic element (not shown) and a processing element (not shown). The downward pressure is positively correlated with, i.e., directly proportional to, the distance by which the elastic elements is compressed and shortened. Furthermore, the processing element generates the pressure correction signal Spc in accordance with the distance by which the elastic elements is compressed and shortened.

Therefore, the elastic elements of the test electrodes 121 get compressed under the downward pressures exerted by the test electrodes 121 upon the acupoint testing points 410, respectively, as soon as the test electrodes 121 come into contact with the acupoint testing points 410 of the human body part 400, respectively. The processing elements of the test electrodes 121 generate the pressure correction signal Spc in accordance with the distance by which the elastic elements is compressed and shortened and then send the pressure correction signal Spc to the processing module 140 so as to correct and turn the impedance signals Si into the correction impedance signals Si′.

During the aforesaid examination process, the downward pressure varies with impedance, and thus the impedance signals Si thus gathered are susceptible to distortion. To eliminate the distortion, the pressure sensing modules 122 generate the pressure correction signals Spc for use in correcting the impedance signals Si, so as to enhance the precision of impedance signal Si (corresponding to the correction impedance signal Si′) detected and measured at the acupoint testing points 410.

Referring to FIG. 5 and FIG. 6, there are shown a function block diagram and a perspective view of the wearable medical examination and treatment system 1000 according to the first aspect of an embodiment of the present invention, respectively. As shown in the diagrams, the wearable medical examination and treatment system 1000 is in wireless communication with the external electronic device 200. As shown in FIG. 5 and FIG. 6, in the first aspect of this embodiment of the present invention, the wearable medical examination and treatment system 1000 comprises the wearable medical examination and treatment device 100 and the cloud server 300. In the second aspect (not shown) of this embodiment of the present invention, the wearable medical examination and treatment system 1000 comprises the wearable medical examination and treatment device 100′ (not shown) and the cloud server 300.

With the wearable medical examination and treatment devices 100, 100′, impedance signals Si pertaining to a subject's physiological state are generated in accordance with a testing instruction Cd and sent to the electronic device 200, and treating electrical signals Sc are generated in accordance with a treating instruction Cc and sent to stimulate the acupoint testing points 410 of the subject. Then, the cloud server 300 receives the impedance signals Si through the electronic device 200 and then converts the impedance signals Si into a physiological information Ip to be displayed on the display module of the electronic device 200. This embodiment is substantially identical to the preceding embodiment in terms of how data is transmitted between the wearable medical examination and treatment device 100, 100′, the electronic device 200 and the cloud server 300 and how conversion of related signals occurs.

In conclusion, the present invention provides a wearable medical examination and treatment device and a wearable medical examination and treatment system which comprise an enclosing unit, a test electrode unit, a ground electrode, a processing module, and a transmission module to thereby simultaneously detect and measure impedance signals at multiple acupoint testing points and thus obtain multiple pieces of physiological information pertaining to the acupoint testing points of a subject, thereby enhancing the efficiency of examination. Once the physiological information is generated and determined to be abnormal, it is feasible to treat the subject in accordance with the physiological information conveniently. Furthermore, the test electrode unit of the wearable medical examination and treatment device further comprises the pressure sensing modules for detecting the impedance signals accurately and thus enhancing the accuracy in examination and diagnosis.

The present invention is disclosed above by preferred embodiments. However, persons skilled in the art should understand that the preferred embodiments are illustrative of the present invention only, but should not be interpreted as restrictive of the scope of the present invention. Hence, all equivalent modifications and replacements made to the aforesaid embodiments should fall within the scope of the present invention. Accordingly, the legal protection for the present invention should be defined by the appended claims.

What is claimed is:

1. A wearable medical examination and treatment device, comprising:
an enclosing unit having a plurality of positioning holes penetrating the enclosing unit, wherein the enclosing unit encloses a human body part of a subject and is fixed to the human body part by the positioning holes;
a test electrode unit comprising a plurality of test electrodes located at predetermined acupoint testing positions in the enclosing unit, respectively, wherein the predetermined acupoint testing positions are aligned with acupoint testing points of the human body part such that the test electrodes are in contact with the acupoint testing points of the human body part, respectively;
a ground electrode disposed in the enclosing unit and adapted to come into contact with the human body part;
a processing module disposed at the enclosing unit, electrically connected to the test electrode unit and the ground electrode, adapted to generate a testing electrical signal in accordance with a testing instruction and send the testing electrical signal to the test electrode unit such that the test electrode unit, the human body part and the ground electrode together form a circuit loop for use in testing impedance signals of the acupoint testing points, and adapted to generate a treating electrical signal in accordance with a treating instruction and send the treating electrical signal to the test electrode unit to therefore stimulate the acupoint testing points; and
a transmission module disposed at the enclosing unit, electrically connected to the processing module, and adapted to send the impedance signals to an electronic device disposed outside such that the electronic device displays a physiological information of the subject in accordance with the impedance signals.

2. The wearable medical examination and treatment device of claim 1, wherein the enclosing unit is glove-shaped and corresponds in shape to the human body part exemplified by a hand, wherein the glove-shaped enclosing unit encloses the hand but allows fingers of the hand to penetrate the positioning holes of the glove-shaped enclosing unit, respectively, and thus be exposed from the enclosing unit.

3. The wearable medical examination and treatment device of claim 1, wherein the enclosing unit is sock-shaped and corresponds in shape to the human body part exemplified by a foot, wherein the sock-shaped enclosing unit encloses the foot but allows portions of the foot to penetrate the positioning holes of the sock-shaped enclosing unit, respectively, and thus be exposed from the enclosing unit.

4. The wearable medical examination and treatment device of claim 1, wherein the test electrode unit further comprises a plurality of pressure sensing modules connected to the test electrodes, respectively, and adapted to sense a downward pressure when in contact with the acupoint testing points and send a pressure correction signal corresponding to the downward pressure to the processing module so as to correct the impedance-related data.

5. The wearable medical examination and treatment device of claim 1, wherein the test electrodes are replaceable button electrodes.

6. The wearable medical examination and treatment device of claim 1, wherein the transmission module is a wireless transmitter.

7. The wearable medical examination and treatment device of claim 1, wherein the acupoint testing points correspond in position to Twelve Source Points described in Yellow Emperor's Inner Canon, respectively.

8. A wearable medical examination and treatment system comprising the wearable medical examination and treatment device of claim 1, the system having wireless communication with an electronic device disposed outside, the wearable medical examination and treatment system comprising:
the wearable medical examination and treatment device for generating an impedance signal related to a physiological state of a subject in accordance with a testing instruction, sending the impedance signal to the electronic device, generating a treating electrical signal in accordance with a treating instruction, and sending the treating electrical signal to stimulate acupoint testing points of the subjects; and
a cloud server for receiving the impedance signals through the electronic device, converting the impedance signals into a physiological information, and sending the physiological information to the electronic device so as for the physiological information to be displayed on a display module of the electronic device.
